(19) United States
(12) Patent Application Publication
(10) Pub. No.: US 2019/0216567 A1
(43) Pub. Date: Jul. 18, 2019

(54) CONTAINER FOR THE STERILE PACKAGING OF ARTICLES UNDER REDUCED PRESSURE

(71) Applicant: Linhardt GmbH & Co. KG, Viechtach (DE)

(72) Inventor: Johann Beil, Lam (DE)

(73) Assignee: Linhardt GmbH & Co. KG, Viechtach (DE)

(21) Appl. No.: 16/304,593

(22) PCT Filed: Mar. 8, 2018
(86) PCT No.: PCT/EP2018/055791

§ 371 Date: Nov. 26, 2018

(30) Foreign Application Priority Data
Mar. 16, 2017 (DE) 10 2017 105 659.9

(51) Int. Cl. A61B 50/30; A61L 2/26

(52) U.S. Cl. CPC A61B 50/30; A61B 2050/006; A61L 2/26

(57) ABSTRACT

The invention relates to a container for sterilely packaging objects under reduced pressure, said container comprising a lower part and an upper part. The container has an annular sealing element having a main sealing-element plane DH, the sealing element being H-shaped in cross-section perpendicular to the main sealing-element plane, with two flanges and a web. An entire lower edge of the upper part and an entire upper edge of the lower part can be slid between the flanges of the sealing element. The sealing element is designed to tightly close the container, the flange of the sealing element arranged on the outside of the container when the container is assembled being equipped with at least one predetermined rupture feature for destroying the tight closure of the container.

### CROSS-REFERENCE TO RELATED APPLICATION

This application claims priority from German patent application No. 102017105659.9 filed Mar. 16, 2017, the disclosure of which is hereby incorporated herein in its entirety by reference.

### FIELD OF THE INVENTION

The invention relates to a container for the sterile packaging of articles under reduced pressure.

### BACKGROUND OF THE INVENTION

Containers for packaging and storing articles under sterile conditions are known from the prior art. Commonly, the sterile conditions are maintained over a longer period in that the articles are stored under reduced pressure.

In this context, U.S. Pat. No. 4,149,650 discloses a storage container for articles and tools that have to be stored under sterile conditions. The container comprises a lower part, as well as a lid with a sealing strip. The container is filled under a vacuum such that the lower part and the lid are subsequently held together due to the external air pressure. A ventilation valve is arranged on the upper side of the lid enabling to neutralize the vacuum in the interior of the container such that the container can be opened and the tools or instruments located therein can be removed. The sealing strip consists of a heat-shrinkable plastic that is shrunk over the valve.

DE 33 29 941 A1 discloses a container that is constructed from multiple parts and serves for respectively packaging and storing foods. The container is provided with a valve, through which a vacuum can be applied by means of a vacuum pump. A tear-off foil is arranged above the valve and can be removed in order to neutralize the vacuum. The container comprises two circumferential sealing elements, which in a cross section have an H-shaped profile.

US 2016/0106507 A1 concerns a packaging for medical articles, preferably medical implants, which consists of two shells that are connected by means of a hinge and placed on top of one another. In the closed state, a sealing strip is arranged over the entire length of the edges of the shells. The sealing strip serves for sealing the two shells relative to one another such that the contents of the packaging can be respectively stored and transported in a sterile state. No additional closure elements are provided for sealing the container. The shrunk-on sealing strip is removed in order to open the container and to remove the articles located therein.

Documents DE 10 2006 034 527 A1 and DE 26 51 291 A1 disclose sterile containers that are constructed from multiple parts and comprise an upper part, a lower part, a vacuum seal or a vacuum lock, as well as sealing elements. In DE 10 2006 034 527 A1, the lid is locked on the lower part during the sterilization process. Mechanical locks are not required because the two container halves are firmly connected to one another due to the vacuum generated within the sterile container. In DE 26 51 291 A1, a vacuum is applied to a container during a sterilization process, e.g. within an autoclave. The container comprises a one-way valve that closes toward the end of the sterilization process. The valve is held open by means of a respectively softening or melting closure element until a sufficiently high temperature is reached and the valve closes. The valve can be manually opened and articles can subsequently be removed from the container.

Despite these solution concepts known from the prior art, there is still a need for containers for the sterile packaging of articles under reduced pressure, which have an utmost simple design and, in particular, can be quickly and easily opened thereby destroying the vacuum in the container.

### SUMMARY OF THE INVENTION

This is the approach of the invention. It aims provide a container for the sterile packaging of articles under reduced pressure, which has a simple design and can be quickly and easily opened while destroying the vacuum in the container. According to the invention, this problem is solved with the container according to independent claim 1. Other advantageous aspects, details and embodiments of the invention can be gathered from the dependent claims, the description, as well as the drawings.

The present invention provides a container for the sterile packaging of articles under reduced pressure. The container comprises a lower part and an upper part. The lower part is constructed from a bottom and at least a lower part sidewall, wherein the at least one lower part sidewall extends upwardly from the bottom and the bottom and the at least one lower part sidewall jointly define a lower part interior with an open upper end. The upper part is constructed from a lid and at least an upper part sidewall, wherein the at least one upper part sidewall extends downwardly from the lid and the lid and the at least one upper part sidewall jointly define an upper part interior with an open lower end. The container also comprises an annular sealing element with a sealing element main plane, wherein the sealing element, in a cross section perpendicular to the sealing element main plane, is realized in an H-shaped manner with two flanges and a web. The upper part is designed such that it can be inserted between the flanges of the sealing element with the entire edge region of the upper part sidewall located adjacent to the open lower end of the upper part and the lower part is designed such that it can be inserted between the flanges of the sealing element with the entire edge region of the lower part sidewall located adjacent to the open upper end of the lower part. The sealing element is designed for tightly sealing the container, wherein the flange of the sealing element, which in the assembled state of the container is arranged on an outer side of the container, is provided with at least one predetermined rupture feature for destroying the tight seal of the container.

After the assembly of the container by using the upper part, the lower part and the sealing element, one flange of the H-shaped sealing element is arranged in the container interior and one flange of the H-shaped sealing element is arranged on the outer side of the container and partially abuts against the upper part sidewall, as well as the lower part sidewall. According to the invention, this flange of the sealing element, which is arranged on the outer side of the container and which is therefore freely accessible, is provided with a predetermined rupture feature for destroying the tight seal of the container. In addition to its primary function of tightly sealing the container, particularly in a gas-tight manner, the sealing element therefore also forms an opening aid that makes it possible to open the container without the prior actuation of a ventilation valve. The opening process can be easily realized because the sealing element loses its sealing function due to the destruction of the predetermined rupture feature and therefore air is admitted into the container until complete or at least approximately complete pressure compensation has taken place. The lower part and the upper part can then be separated from one another with little effort.

It is particularly preferred that the upper part and the lower part are realized in one piece. In this case, no preassembly of the lid and the upper part sidewall or the bottom and the lower part sidewall is respectively required. In particularly preferred embodiments, the upper part and the lower part also respectively consist of a single, one-piece sidewall. In this case, no preassembly of multiple sidewalls is required for respectively forming the upper part interior and the lower part interior.

According to a preferred embodiment, the predetermined rupture feature for destroying the tight seal of the container consists of at least one perforation that extends over the entire vertical extent of the sealing element flange that is oriented perpendicular to the sealing element main plane, which in the assembled state of the container is arranged on the outer side of the container. A perforation of the sealing element flange arranged on the outer side of the container forms a predetermined rupture feature that can be very easily produced and actuated without any difficulty. It shall be explicitly noted that only the sealing element flange arranged on the outer side of the container is perforated in the described embodiment, i.e. not the web and not the flange located in the container interior. In this way, the sealing function of the sealing element continues to be ensured despite the provided perforation.

The predetermined rupture feature for destroying the tight seal of the container is advantageously provided with a notch that enables to tear open the sealing element more easily. This process can be realized in a particularly simple manner if such an initial tearing notch is operatively connected to the perforation of the sealing element flange arranged on the outer side of the container.

According to another particularly preferred embodiment of the present invention, the sealing element flange, which in the assembled state of the container is arranged on the outer side of the container, comprises a tab-like extension, wherein the tab-like extension is operatively connected to the predetermined rupture feature of the sealing element. It is furthermore particularly preferred to provide an initial tearing notch that is operatively connected to the predetermined rupture feature. Due to the tab-like extension and optionally also the initial tearing notch, the sealing element can be gripped in a particularly simple manner without any difficulties. Since the tab-like extension is operatively connected to the predetermined rupture feature and optionally also to the initial tearing notch of the sealing element, the predetermined rupture feature can be destroyed by simply pulling on the tab-like extension such that the sealing element loses its sealing function and the upper part and the lower part of the container can subsequently be separated from one another without any difficulties. This embodiment is particularly advantageous if medical products, particularly surgical instruments, are stored in the container. In operating rooms, it frequently occurs that the person, who should open the container, only has one hand available for opening the container. In the present embodiment, the tab formed on the sealing element can be designed in such a way that the predetermined rupture feature can be destroyed with little practice by taking hold of the tab with the thumb and index finger or by a translatory motion of the thumb while the container is clamped between two fingers of the same hand. The container can be altogether opened with one hand by subsequently flipping over the upper part with the aid of the thumb.

The upper part and the lower part of the container should be made of the same material. For example, plastics may be used as wall material. It is particularly preferred that the lower part and the upper part are made of aluminum, an aluminum alloy or steel. Aluminum can be easily formed and is particularly suitable for the production of various container shapes.

The bottom, the at least one lower part sidewall, the lid and the at least one upper part sidewall preferably have a wall thickness of 0.2 mm to 1.5 mm, particularly 0.5 mm to 1.0 mm, especially around 0.7 mm. The cited wall thicknesses on the one hand allow a material-saving production and on the other hand provide the container with a sufficient dimensional stability for withstanding the pressure differential between the container interior and the surrounding atmosphere after the container has been filled under a vacuum. It is particularly preferred that the bottom, the at least one lower part sidewall, the lid and the at least one upper part sidewall have the same wall thickness.

If the bottom, the lower part sidewall, the lid and the upper part sidewall are made of the same material and have the same wall thickness, the tightness of the container is also ensured in case of a potentially occurring minimal deformation of the upper part or the lower part as a result of the pressure differential between the container interior and the surroundings.

The at least one lower part sidewall and/or the at least one upper part sidewall is preferably provided with at least one reinforcing rib. The reinforcing rib may be realized in the form of an elevation or depression in the lower part sidewall or the upper part sidewall and is stamped into the respective sidewall with constant wall thickness. The reinforcing rib may have a depth of around 2 mm, which may be constant or vary over the extent of the reinforcing rib. The depth of the reinforcing rib particularly may decrease to zero towards the edge of the front or rear side of the wall, respectively. The at least one reinforcing rib additionally improves the dimensional stability of the container such that it can also withstand higher pressure differentials between the container interior and the surrounding atmosphere after having been filled under a vacuum. The lower part sidewall, as well as the upper part sidewall, may be advantageously provided with multiple reinforcing ribs.

According to an advantageous embodiment, a vertical extent of the sealing element web parallel to the flanges is between 1 mm and 10 mm, preferably between 1.5 mm and 6 mm, particularly around 2 mm. The web is advantageously dimensioned in such a way that it cannot be penetrated by the edge regions of the upper part sidewall and the lower part sidewall during their insertion between the sealing element flanges and that a predetermined rupture feature, e.g. a perforation, does not lead to a loss of the sealing function of the sealing element.

According to another advantageous embodiment, a horizontal extent of the sealing element web perpendicular to the flanges is between 1 mm and 8 mm, preferably between 2 mm and 5 mm, particularly at 3 mm. The horizontal extent of the sealing element web is advantageously adapted to the wall thickness of the upper part and the lower part.

A vertical extent of the sealing element flanges perpendicular to the sealing element main plane preferably is between 5 mm and 50 mm, particularly between 10 mm and 30 mm, especially around 15 mm. Starting from the web, the flanges have to extend upward and downward by a certain distance in both directions in order to ensure the tightness of the container, as well as to preclude the container from being inadvertently opened. The extent of the flanges in the vertical direction perpendicular to the sealing element main plane can generally be adapted to the extent of the upper part or the lower part, respectively. In this context, it has to be observed that the extent of the flanges has to increase with an increasing extent of the container.

The sealing element may basically be made of any conventional materials used for seals. The sealing element is preferably made of plastic, rubber or silicone.

It shall be clarified that the upper part and the lower part of the container may basically have any shape. The horizontal cross section of the container may altogether have an elliptical shape, a rectangular shape or a circular shape. Any irregularly shaped containers would also be conceivable. It is particularly preferred that the upper part and the lower part have an identical horizontal cross-section. Different shapes of the upper part and the lower part can only be compensated with an elaborate adaptation of the design of the H-shaped sealing element.

In particularly preferred containers, the shape and the extent of the bottom and of the lid are essentially identical, the at least one upper part sidewall and the lid include an angle of around 90° with one another and the at least one lower part sidewall and the bottom include an angle of around 90° with one another.

The present invention also pertains to the use of an above-described container for the sterile packaging of medical products under reduced pressure. As already mentioned above, the present invention provides the option of making available a container for storing medical products, particularly surgical instruments, which can be opened with one hand despite the pressure differential between the container interior and the external surroundings. In operating rooms, in particular, it frequently occurs that a person only has one hand available for opening the container. The utilization of an above-described container for the sterile packaging of medical products under reduced pressure is particularly advantageous in these circumstances. In particularly advantageous embodiments, the upper part and the lower part are dimensioned in such a way that the article stored in the container remains in one of the two parts after the container has been opened, protrudes from the respective part and therefore can be easily removed.

The present invention furthermore relates to a method for the sterile packaging of articles under reduced pressure with the steps of
a) supplying an above-described container for the sterile packaging of medical products under reduced pressure in the non-assembled state,
b) introducing the lower part, the upper part, the sealing element and the at least one article to be packaged into an environment under reduced pressure,
c) introducing the at least one article to be packaged into the lower part interior or the upper part interior,
   d1) inserting the edge region of the upper part sidewall located adjacent to the open lower end of the upper part between the flanges of the sealing element,
   d2) inserting the edge region of the lower part sidewall located adjacent to the open upper end of the lower part between the flanges of the sealing element, and
e) increasing the environmental pressure to the normal pressure,
wherein at least either step d1) or step d2) is carried out after step b),
wherein at least either step d1) or step d2) is carried out after step c),
wherein step e) is carried out after steps c), d1) and d2), and
wherein the insertion in steps d1) and d2) takes place in such a way that the predetermined rupture feature of the sealing element is in the assembled state of the container arranged on an outer side of the container.

In the inventive method, the upper part or the lower part can already be inserted between the sealing element flanges prior to the introduction into an environment under reduced pressure. However, all individual parts can also be separately introduced into the environment under reduced pressure prior to connecting the upper part or the lower part to the sealing element. It should be obvious that the insertion of the second part and therefore sealing of the container does not take place until the article to be packaged has been introduced into the lower part interior or the upper part interior. It is also absolutely imperative that the insertion of the upper part and the lower part between the sealing element flanges takes place in such a way that the predetermined rupture feature is in the assembled state of the container arranged on an outer side of the container and therefore accessible for the user.

Enhancements, advantages and potential applications of the invention can also be gathered from the following description of exemplary embodiments and the figures.

### BRIEF DESCRIPTION OF THE DRAWINGS

An exemplary embodiment of the invention is described in greater detail below with reference to the drawings. In these drawings,

FIG. 1 schematically shows a perspective view of an inventive container;

FIG. 2 schematically shows a side view of the container according to FIG. 1 with a portion including a sealing element shown in cross-section;

FIG. 3 schematically shows another side view of the container according to FIG. 1 in cross-section; and

FIG. 4 schematically shows a detail of FIG. 2 in the form of an enlarged representation in cross-section.

### DETAILED DESCRIPTION OF THE INVENTION

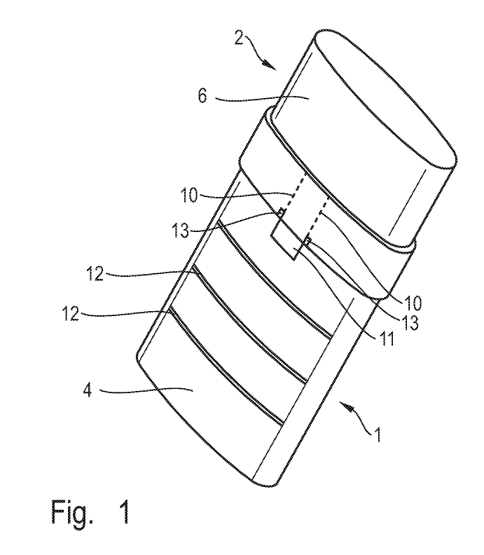

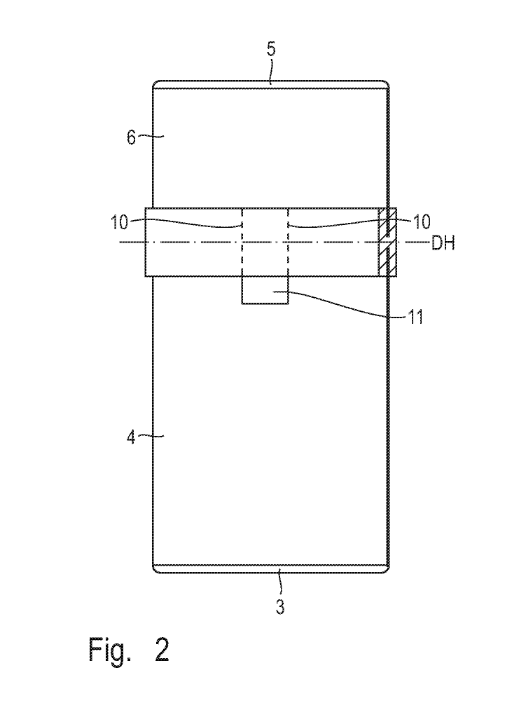

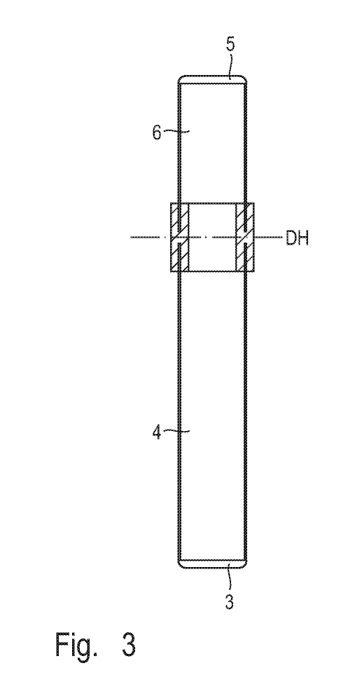

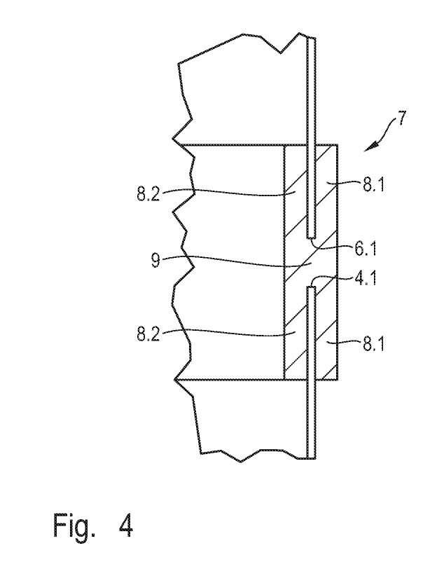

FIGS. 1 to 4 schematically show different views of an inventive container for the sterile packaging of articles under reduced pressure.

The container comprises a lower part 1 and an upper part 2. The lower part 1 is realized in one piece and constructed from a bottom 3 and a lower part sidewall 4, wherein the lower part sidewall 4 extends vertically upward from the bottom 3 and the bottom 3 and the lower part sidewall 4 jointly define a lower part interior with an open upper end. The upper part 2 is likewise realized in one piece and constructed from a lid 5 and an upper part sidewall 6, wherein the upper part sidewall 6 extends vertically downward from the lid 5 and the lid 5 and the at least one upper part sidewall 6 jointly define an upper part interior with an open lower end.

The upper part 2 and the lower part 1 of the container are made of aluminum. The bottom 3, the lower part sidewall 4, the lid 5 and the upper part sidewall 6 have a wall thickness of around 0.7 mm. The lower part sidewall 4 is provided with three reinforcing ribs 12 (only illustrated in FIG. 1). The cited wall thickness and the reinforcing ribs on the one hand allow a material-saving production and on the other hand provide the container with a sufficient dimensional stability for also withstanding the pressure differential between the container interior and the surrounding atmosphere after the container has been filled under a vacuum without any difficulties.

The container also comprises an annular sealing element 7 with a sealing element main plane DH, wherein the sealing element 7, in a cross section perpendicular to the sealing element main plane DH is realized in an H-shaped manner with two flanges 8.1, 8.2 and a web 9. The upper part 2 is inserted between the flanges 8.1, 8.2 of the sealing element 7 with the entire edge region 6.1 of the upper part sidewall 6 located adjacent to the open lower end of the upper part and the lower part 2 is inserted between the flanges of the sealing element with the entire edge region 4.1 of the lower part sidewall 4 located adjacent to the open upper end of the lower part. The sealing element 7 made of silicone tightly seals the container. The flange 8.1 of the sealing element 7 arranged on the outer side of the container is provided with two predetermined rupture points features 10 and two initial tearing notches 13, which respectively are operatively connected to a predetermined rupture feature 10, in order to destroy the tight seal of the container. The predetermined rupture features 10 are realized in the form of a perforation of the flange 8.1.

The flange 8.1 of the sealing element 7 arranged on the outer side of the container comprises a tab-like extension 11, wherein the tab-like extension 11 is operatively connected to the two predetermined rupture features 10 of the sealing element 7. The tab-like extension 11 makes it possible to grip the sealing element in a particularly simple manner without any difficulties. Since the tab-like extension 11 is operatively connected to the predetermined rupture features 10 of the sealing element 7, the predetermined rupture features 10 can be destroyed by simply pulling on the tab-like extension 11 such that the sealing element 7 loses its sealing function and the lower part 1 and the upper part 2 of the container can subsequently be separated from one another without any problems. The tab 11 formed on the sealing element 7 is designed in such a way that the predetermined rupture features 10 can be destroyed by taking hold of the tab 11 with the thumb and index finger or by a translatory motion of the thumb while the container is clamped between two fingers of the same hand. The container can be altogether opened with one hand by subsequently flipping over the upper part with the aid of the thumb.

A vertical extent of the web 9 of the sealing element 7 parallel to the flanges 8.1, 8.2 lies around 4 mm. The web is dimensioned in such a way that it is not penetrated by the edge regions 6.1, 4.1 of the upper part sidewall 6 and the lower part sidewall 4 during their insertion between the flanges 8.1, 8.2 of the sealing element 7. At the same time, the perforations 10 cannot lead to a loss of the sealing function of the sealing element 7.

The horizontal extent of the web 9 of the sealing element 7 perpendicular to the flanges 8.1, 8.2 amounts to 3 mm and is therefore well adapted to the wall thickness of the edge regions 6.1, 4.1 of the upper part sidewall 6 and the lower part sidewall, which respectively amounts to around 0.7 mm.

A vertical extent of the flanges 8.1, 8.2 of the sealing element 7 perpendicular to the sealing element main plane DH lies around 20 mm. Starting from the web, the flanges extend upward and downward by a distance of 8 mm in both directions such that the tightness of the container is ensured and the container is also precluded from being inadvertently opened. The vertical extent of the flanges 8.1, 8.2 perpendicular to the sealing element main plane DH is therefore respectively well adapted to the extent of the upper part 2 of around 45 mm and the extent of the lower part 1 of around 95 mm.

### LIST OF REFERENCE SYMBOLS

1 Lower part
2 Upper part
3 Bottom
4 Lower part sidewall
4.1 Edge region of lower part sidewall
5 Lid
6 Upper part sidewall
6.1 Edge region of upper part sidewall
7 Sealing element
8.1, 8.2 Flanges of sealing element
9 Web of sealing element
10 Predetermined rupture feature
11 Tab-like extension
12 Reinforcing ribs
13 Initial tearing notch
DH Sealing element main plane

What is claimed is:

1. A container for the sterile packaging of articles under reduced pressure, wherein the container comprises a lower part and an upper part, wherein the lower part is constructed from a bottom and at least a lower part sidewall, wherein the at least one lower part sidewall extends upwardly from the bottom and wherein the bottom and the at least one lower part sidewall jointly define a lower part interior with an open upper end, wherein the upper part is constructed from a lid and at least an upper part sidewall, wherein the at least one upper part sidewall extends downwardly from the lid and wherein the lid and the at least one upper part sidewall jointly define an upper part interior with an open lower end, wherein the container comprises an annular sealing element with a sealing element main plane, wherein the sealing element in a cross section perpendicular to the sealing element main plane (DH) is realized in an H-shaped manner with two flanges and a web, wherein the upper part is designed such that it can be inserted between the flanges of the sealing element with an entire edge region of the upper part sidewall located adjacent to the open lower end of the upper part and the lower part is designed such that it can be inserted between the flanges of the sealing element with an entire edge region of the lower part sidewall located adjacent to the open upper end of the lower part, wherein the sealing element is designed for tightly sealing the container, and wherein the flange of the sealing element, which in an assembled state of the container is arranged on an outer side of the container, is provided with at least one predetermined rupture feature for destroying the tight seal of the container.

2. The container according to claim 1, characterized in that the predetermined rupture feature for destroying the tight seal of the container consists of at least a perforation that extends over the entire vertical extent of the flange of the sealing element that is oriented perpendicular to the sealing element main plane, which in the assembled state of the container is arranged on the outer side of the container.

3. The container according to claim 1, characterized in that the flange of the sealing element, which in the assembled state of the container is arranged on the outer side of the container, comprises a tab-like extension, wherein the tab-like extension is operatively connected to the predetermined rupture feature of the sealing element.

4. The container according to claim 1, characterized in that at least one initial tearing notch is provided and operatively connected to the predetermined rupture feature.

5. The container according to claim 1, characterized in that the lower part and/or the upper part consists of aluminum, an aluminum alloy or steel.

6. The container according to claim 1, characterized in that the bottom, the at least one lower part sidewall, the lid and the at least one upper part sidewall have a wall thickness of 0.2 mm to 1.5 mm, preferably 0.5 mm to 1.0 mm, particularly preferably around 0.7 mm.

7. The container according to claim 1, characterized in that the at least one lower part sidewall and/or the at least one upper part sidewall is provided with at least one reinforcing rib.

8. The container according to claim 1, characterized in that a vertical extent of the web of the sealing element parallel to the flanges is between 1 mm and 10 mm, preferably between 1.5 mm and 6 mm, particularly preferably around 2 mm.

9. The container according to claim 1, characterized in that a horizontal extent of the web of the sealing element perpendicular to the flanges is between 1 mm and 8 mm, preferably between 2 mm and 5 mm, particularly preferably at 3 mm.

10. The container according to claim 1, characterized in that a vertical extent of the flanges of the sealing element perpendicular to the sealing element main plane is between 5 mm and 50 mm, preferably between 10 mm and 30 mm, particularly preferably around 15 mm.

11. The container according to claim 1, characterized in that the sealing element consists of plastic, rubber or silicon.

12. The container according to claim 1, characterized in that
the shape and the extent of the bottom and of the lid are essentially identical,
the at least one upper part sidewall and the lid include an angle of around 90° with one another and
the at least one lower part sidewall and the bottom include an angle of around 90° with one another.

13. Use of a container according to claim 1 for the sterile packaging of medical products under reduced pressure.

14. A method for the sterile packaging of articles under reduced pressure comprising the steps of
a) supplying a container according to claim 1 in a non-assembled state,
b) introducing the lower part, the upper part, the sealing element and the at least one article to be packaged into an environment under reduced pressure,
c) introducing the at least one article to be packaged into the lower part interior or the upper part interior,
d1) inserting the edge region of the upper part sidewall located adjacent to the open lower end of the upper part between the flanges of the sealing element,
d2) inserting the edge region of the lower part sidewall located adjacent to the open upper end of the lower part between the flanges of the sealing element, and
d) increasing the environmental pressure to the normal pressure, wherein at least either step d1) or step d2) is carried out after step b), wherein at least either step d1) or step d2) is carried out after step c), wherein step e) is carried out after steps c), d1) and d2), and wherein the insertion in steps d1) and d2) takes place in such a way that the predetermined rupture feature of the sealing element (7) is in the assembled state of the container arranged on an outer side of the container.
